(19) United States
(12) Patent Application Publication
(10) Pub. No.: US 2004/0115180 A1
(43) Pub. Date: Jun. 17, 2004

(54) Carrier chimeric proteins targeted carrier chimeric proteins and preparation thereof

(76) Inventor: Mustapha Abdelouahed, Worchester, MA (US)

Correspondence Address: Traskbritt, PO Box 2550, Salt Lake City, UT, 84110, US

(21) Appl. No.: 10/469,613

(22) PCT Filed: Mar. 6, 2002
(86) PCT No.: PCT/US02/06882

(51) Int. Cl. A61K 38/43; C12N 9/16

(52) U.S. Cl. 424/94.1; 435/196

(57) ABSTRACT

A chimeric carrier protein having a multimerization domain and at least one drug attached thereto via a spacer is disclosed. The protein may be targeted by associating at least one amino acid sequence having an amino acid domain targeted to a specific site of action. In a further embodiment of the invention a nucleic acid molecule is provided which encodes the protein. Vectors containing the nucleic acid molecule and the host cells containing the vectors may also be provided. A method for producing the carrier chimeric protein on the targeted carrier chimeric protein is also disclosed.

### PRIORITY CLAIM

This application claims the benefit of the filing date of U.S. Provisional Patent Application Serial No. 60/273,573, filed Mar. 6, 2001, for “CARRIER CHIMERIC PROTEINS, TARGETED CARRIER CHIMERIC PROTEINS AND PREPARATION THEREOF”.

### TECHNICAL FIELD

(a) Field of the Invention

The invention relates to carrier chimeric proteins comprising a protein- or amino acid-drug, with or without a specific amino acid domain for a specific site of action, methods suitable for their preparation and uses in therapy.

(b) Description of Prior Art

Drug delivery system

### BACKGROUND

In recent years, the development of a drug delivery system (DDS) that maximizes the drug effect and minimizes the side effects has been sought. DDS can be classified according to morphology and methods of administration as follows: (i) A system in which a drug is complexed to a polymeric membrane or formed as a molded product and is adhered on skin or a mucous membrane for slow release or absorption of the drug through the skin or the mucous membrane, respectively. (ii) A body implant system in which a drug complexed to various forms of matrix is left in an organ or subcutaneous tissues for slow release. And (iii) a system in which a drug microencapsulated by means of liposome or lipid microspheres or a prodrug formed by covalently bonding a drug to a polymeric compound is administered directly in blood or tissues.

As an example of the body implant system, described in (ii) above, an anticancer agent is complexed to a polymeric carrier. The implant is applied to the cancerous host and the anti-cancer agent is released continuously. The implant has been developed to reduce the size of tumor, extend the life and relieve pain caused by the cancer. This system has been applied to drugs other than anticancer agents, for example anesthetics, narcotic antagonists, immunoactivators such as interleukin, and interferons, and various hormones. In body implants, a drug is dispersed in polymeric matrix mainly by physical means, and allowed to diffuse from the interior of the matrix to carry out a slow release. Because certain drugs can be readily complexed to the matrix, the technology is applicable to a broad range of drugs. Another advantage of the system is that there is very little loss of activity of the drug during the manufacturing process. The clinical application of these body implants requires implantation by a surgical means in a form suitable for its application, such as needles, rods, films or pellets. The polymeric matrix can include polymers that do or do not degrade in the body. In the case of a matrix that does not decompose in the body, the implant has to be extracted by surgical method after releasing the drug contained therein. Thus, the implants that must be removed surgically are not desirable for clinical application because of pain, infection, and scar formation that might be imposed on the patient. Additionally, the action of the drug being released from an implant left in the body tends to be limited to the region in contact with the implant. Therefore, distribution of the drug in the focus region tends to be non-uniform. Further, an implant embedded in the body may act as an antigen. The implant may be recognized by the body as a foreign substance, and a capsulation consisting of the tissue components is formed around the implant as a defense mechanism. As a result, the efficiency of delivery of the drug to the focus is reduced further. Thus, the body implant system has numerous problems.

Drugs microencapsulated in liposome or lipid microspheres as described in (iii) above are being developed in an effort to overcome the problems associated with body implants. Microencapsulated drugs can be administered directly into blood or tissues without requiring surgical treatment. Certain products of this type are being developed and used clinically. Examples are oil-soluble drugs such as steroids, indomethacin, prostaglandin and so on, mixed into lipid microspheres, and water-soluble anti-cancer drugs such as adriamycin or mitomycin or water-soluble hormones such as insulin, microencapsulated in a liposome. The lipid microsphere is a droplet of soybean oil, coated with a monolayer film of lecithin. Therefore, this application is only useful for drugs that are soluble in soybean oil, and not useful for water-soluble drugs. Also, because lipid microspheres are prepared by suspending soybean oil and lecithin in water, particle size is large and uneven, and thus it is difficult for the product to be distributed uniformly and broadly when it is injected into tissue. Further, the drug being incorporated in lipid microspheres is released by a diffusion process through the oil droplet. Thus, the rate of release decreases exponentially, and continuous release at a constant rate is difficult. Similar to the situation with lipid microspheres, it is difficult to manufacture liposome products with a uniform particle size and to achieve a uniform or broad distribution of the drug when injected in the tissues. Also, there are problems with stability during storage and mechanical strength of the product, making it difficult to maintain the slow-releasing property of the drug for a lengthy period of time. There are problems in the stability of liposomes enveloping an aqueous phase with a lipid bilayer during its storage and in the case of administration into blood, almost all liposomes are taken up into tissue with a developed reticuloendothelial system, such as liver and spleen, so that they are difficult to distribute to other cells or tissues. This is believed to be the case since liposomes have a structure wherein the inner and outer aqueous phases are separated from each other by a phospholipid bilayer and the liposome is thus unstable to various forces. An increase in particle diameter due to aggregation is another known defect during its storage.

Oligomerization Domain

Designed multimeric ligands and inhibitors for multimeric receptors are of high practical use in drug design because of their increased affinity. By protein engineering, oligomerization domains may be artificially linked to functional domains of interest. Engel's group have studied a number of such systems and observed a large increase in thermal stability in a chimera consisting of collagen-like peptides attached to the N-terminus of the foldon domain (Engel & Kamerrer. Matrix Biology, 2000, 19: 283-288). Other effects are multivalency and the increase in intrinsic concentration by oligomerization. Homoassociation of E-cadherin, for example, is not observed for cadherin monomers but is much enhanced in oligomers in which five E-cadherin ectodomains are linked by the coiled coil domain of TSP-5 (Tomschy et al., EMBO J, 1996, 15: 3507-3514).

A classical example for a functionally important oligomerization of binding domains by collagen triple helices is Clq, a subunit of the first component of complement Cl (Kishore & Reid. Immunopharmacology, 1999, 42: 15-21). It is believed that the oligomeric structure of C1q is designed for efficient binding of clusters of IgG at an immunologically marked cell surface, avoiding binding to isolated IgG, which would cause unwanted reactions with the complement system (Tschopp. Mol Immunol, 1982, 19: 651-657). Similar effects of oligomerization may apply to other collagenous molecules (Kishore & Reid. Immunopharmacology, 1999, 42: 15-21) and to collagens containing N- and C-terminal globular extensions. In the type 1 class A macrophage scavenger receptor (Krieger & Herz. Ann Rev Biochem, 1994, 63: 601-637), the globular heads are connected to a collagen triple helix, which is followed by three-stranded coiled coil. The two oligomerization domains probably stabilize each other in a mutual manner. It is known that most lectins recognize monomeric sugars with only weak affinity and polymeric structures with high affinity. In many cases, this physiologically important feature is generated by oligomerization of lectin domains (Engel & Kamerrer. Matrix Biology, 2000, 19: 283-288).

The α-helical coiled coil is probably the most widespread subunit oligomerization motif found in proteins (Engel & Kamerrer. Matrix Biology, 2000, 19: 283-288). Accordingly, coiled coil fulfills a variety of different functions. In several families of transcriptional activators, for example, short leucine zippers play an important role in positioning the DNA-binding regions on the DNA (Ellenberger et al., Cell, 1992, 71: 1223-1237). The leucine zipper domains of Jun and Fos transcription factors comprise 35 amino acid residues that specifically fold into a parallel two-stranded coiled coil heterodimer (Glover & Harisson. Nature, 1995, 373: 257-261). Using insect cells, Eble et al., (Biochemistry, 1998, 37: 10945-10955) expressed large quantities of functional soluble human integrin α3β1 ectodomain heterodimers, in which cytoplasmic and transmembrane domains were replaced by Fos and Jun dimerization motifs. In direct ligand binding assays, soluble α3β1 specifically bound to laminin-5 and laminin-10, and also to invasin, a bacterial surface protein which mediates entry of Yersinia species into the eukaryotic host cell. The functional regulation of the purified soluble integrin α3β1 ectodomain heterodimer by divalent cations resembled that of wild-type membrane-anchored β1 integrin. A soluble T-cell receptor heterodimer was produced for biophysical studies by fusing polypeptide chains corresponding to the constant and variable region of the α and β subunits to the coiled coil domains of Jun and Fos respectively (willox et al., Protein Sci, 1999, 8: 2418-2423). The heterodimeric protein was purified in milligram yields and found to be homogeneous, antibody-reactive, and stable at concentration lower than 1 μM. Based on studies of the Jun, Fos, and GCN4 leucine zippers, O' Shea et al., (Curr Biol, 1993, 3: 658-667) designed a heterodimeric coiled coil termed “Velcro”. This coiled coil has, for example, been used for the expression and functional analysis of ectodomain fragments of CD8αα and CD8αβ dimers (Kern et al., J Biol Chem, 1999, 274: 27237-27243). Wu et al., (Protein Sci, 1999, 8: 482-489) used a designed coiled coil to generate functional soluble forms of both the pseudo-high affinity and the intermediate affinity heterodimeric IL-2 receptors.

When an oligomerization domain is connected to an another multistranded domain, it can stabilize this domain substantially. This was recently demonstrated for a chimera consisting of a collagen-like peptide attached to the N-terminus of foldon. The peptide (gly-pro-pro)₁₀ forms a collagen triple helix, but its thermal stability is very low, and the transition of three randomly coiled chains to the triple helix is highly concentration dependent. The increase in the thermal stability of the collagen triple helix is achieved by the high intrinsic concentration of the C-terminus ends of the collagen chains, which is enforced by the trimeric foldon (Engel & Kamerrer. Matrix Biology, 2000, 19: 283-288). Coiled coils are also used to form oligomers of intermediate filament proteins. The members of this family are important components of the cytoskeleton and form large, mechanically rigid structures such as hair scales and feathers (keratin). Coiled coil proteins furthermore appears to play an important role in both vesicle and viral membrane fusion (Skehel and whey. Cell, 1998, 95: 871-874). In the extracellular space, the heterotrimeric coiled coil protein laminin plays an important role in the formation of basement membranes. Apparently, the multifunctional oligomeric structure is required for laminin function.

Other examples include the thrombospondins in which three (TSP-1 and TSP-2) or five (TSP-3, TSP-4 and TSP-5 (or COMP)) chains are connected. The molecules have a flower bouquet-like appearance, and the reason for their oligomeric structure is probably the multivalent interaction of their domains with cellular receptors (Engel & Kamerrer. Matrix Biology, 2000, 19: 283-288). Interestingly, the five-stranded coiled coil domains contain a hydrophobic channel, which can accommodate vitamins A and D. A potential storage and delivery function for cell signaling molecules has been proposed for the coiled coil domain of COMP (COMPcc) (Guo et at., EMBO J, 1998, 17: 5265-5272). COMPcc comprises 46 residues, which fold into a parallel five-stranded coiled coil (malashkevich et al., Science, 1996, 274: 761-765). The domain has been used to mimic cluster formation of E-cadherin on the cell surface (Tomschy et al., EMBO J, 1996, 15: 3507-3514), a process that is believed to be of major importance for cell-cell adhesion. Electron microscopy, analytical ultracentrifugation, solid phase binding and cell attachment assays revealed a strong self-association and cell attachment of pentamers, whereas monomers exhibited no activity. COMPcc has also been used to design improved soluble inhibitors of FasL and CD40L based on oligomerized receptors (Holler et al., J Immunol Methods, 2000, 237: 159-173). Members of the tumor necrosis factor receptor (TNFR) superfamily have an important role in the induction of cellular signals resulting in cell growth, differentiation or death. TNFR family members fused to the constant domain of immunoglobulin G have been widely used as immunoadhesins in basic in vitro and in vitro research and in some clinical applications. The affinity of Fas and CD40 (but not TNFR-1 and TRAIL-R2) to their ligands is increased by fusion to COMPcc, when compared to the respective Fc chimeras. In functional assays, Fas-COMP was at least 20-fold more active than Fas-Fc at inhibiting the action of sFasL, and CD40-COMP could block CD40L-mediated proliferation of B cells, whereas CD40-Fc could not. Pack et al., (J Mot Biol, 1995, 246: 28-34) have designed tetravalent miniantibodies assembling in the periplasm of E. Coli. They were based on single-chain Fv fragments, connected via a flexible hinge to the four-stranded GCN4p-LI mutant. The affinity of the tetravalent miniantibody was higher in ELISA and BIAcore measurements than that of the bivalent construct.

It would be highly desirable to be provided with a carrier chimeric protein containing a drug attached thereto, which has a higher activity and stability than the drug itself, therefore allowing better medical application, treatment or therapy.

### DISCLOSURE OF INVENTION

One aim of the present invention is to provide a carrier chimeric protein containing a drug attached thereto, which has a higher activity and stability than the drug itself, therefore allowing better medical application, treatment or therapy.

In accordance with the present invention there is provided a chimeric carrier protein comprising a multimerisation domain and at least one drug attached thereto, via a spacer.

Still in accordance with the present invention, there is provided a targeted chimeric carrier protein comprising a multimerisation domain, at least one drug attached thereto, via a spacer, and at least one amino acid sequence having an amino acid domain targeted to a specific site of action.

Further in accordance with the present invention, there is provided a nucleic acid molecule encoding the carrier chimeric protein or the targeted carrier chimeric protein, as well as vectors containing such nucleic acid molecule and host cells containing such vectors.

In accordance with the present invention, there is also provided a method for producing the carrier chimeric protein or the targeted carrier chimeric protein, which comprises maintaining in suitable conditions host cells as defined above for producing the carrier chimeric protein or the targeted carrier chimeric protein.

### BRIEF DESCRIPTION OF DRAWINGS

FIGS. 1A and 1B are schematic representations of one embodiment of the invention using the multimerization domain of TSP-1;

FIG. 2 is a representation of the amino acid sequence of human TSP-1;

FIGS. 3A and 3B are schematic representations of one embodiment of the invention using the multimerization domain of TSP-5;

FIG. 4 is a representation of the amino acid sequence of human TSP-5 (or COMP);

FIGS. 5A and 5B are schematic representations of one embodiment of the invention using the multimerization domain of TSP-5 with different drugs; and

FIGS. 6A and 6B are schematic representations of one embodiment of the invention using the multimerization domain of TSP-1 with different drugs.

### BEST MODE(S) FOR CARRYING OUT THE INVENTION

The present invention relates to carrier chimeric proteins with a protein- or amino acid-drug, with or without a specific amino acid domain to a specific site of action, and conjugation methods suitable for their preparation and use in therapy. These chimeric proteins are assembled into a multimeric structure.

Many proteins form their native structure only in their oligomeric state and are unfolded as monomers. Collagen triple helices and coiled coil structures are examples of such obligatory oligomers, but there are also a number of representatives among globular proteins. Many examples illustrate the fact that oligomerization domains play an important role in protein function. In the extracellular matrix, the induced multivalency is of high importance because of the need for interactions between many partners in large networks. Collagen triple helices, coiled coils and other oligomerization domains mediate the subunit assembly of a large number of proteins. Oligomerization leads to functional advantages of multivalency and high binding strength, increased structure stabilization and combined functions of different domains. These features seen in naturally occurring proteins can be engineered by protein design by combining oligomerization domains with functional domains.

The invention also intends to include carrier chimeric proteins comprising the multimerization domain of any multimeric protein with a coiled coil domain (e.g., dimeric, trimeric, tetrameric or pentameric protein, or analogous products), a protein- or an amino acid-drug, with or without a specific amino acid domain to a specific site of action, or analogous products.

According to one aspect of the present invention, there is provided a new method for attaching a protein- or amino acid-drug, to the multimerization domain of a multimeric protein with a coiled coil domain. The multimerization domain of the multimeric protein with a coiled coil domain can be of variable length, depending on the requirements of the application. The multimerization domain of thrombospondin (TSP) protein (e.g., TSP-1 or TSP-5 (also named cartilage oligomeric matrix protein: COMP)) and other such glycoproteins with a coiled coil domain (including matrilin, laminin, tenascin, collectin, and collagen, among others) can be used to make these chimeric proteins. The expression protein- or amino acid-drug, is intended to mean any peptide-, polypeptide-, protein-drug or conjugate such as a thrombolytic agent (e.g., streptokinase, urokinase, single chain urokinase-like plasminogen activator, prourokinase, or derivatives).

According to another embodiment of the present invention, by inserting as much as desired of the active drug-peptide, a chimeric protein has an increased activity of the protein- or amino acid-drug over the single protein- or amino acid-drug. For example, if the multimerization domain of the protein is trimeric (e.g., TSP-1, and TSP-2), the number of inserted protein- or amino acid drug could be a factor of 3 (3, 6, 9, etc), and if the multimerization domain of the protein is pentameric (e.g., TSP-3, TSP-4 and TSP-5), the number of inserted protein- or amino acid-drug could be a factor of 5 (5, 10, 15, etc).

In such targeted chimeric proteins, one can insert any amino acid motif specific for a site of action of the active-drug-peptide (also called protein- or amino acid-drug). It is an additional (and optional) feature of the present invention that, because an amino acid motif specific for a site of action of the active-drug-peptide is inserted in the chimeric protein, the targeted chimeric protein product acts as a targeting agent. Such targeting agent will be chosen with regard to the site of action, and to the nature of the problem that is to be addressed.

The invention further includes isolated nucleic acids encoding any of the above chimeric proteins, vectors comprising these nucleic acids, and host cells comprising any of said vectors. The present invention further encompasses gene therapy methods whereby DNA sequences encoding carrier chimeric proteins, targeted carrier chimeric proteins or conjugates are introduced into a patient. The selection of the appropriate gene therapy methods to treat a disease will be apparent to one skilled in the art.

Carrier chimeric proteins with a protein- or amino acid-drug, with or without a specific amino acid domain for a specific site of action, and conjugation methods suitable for their preparation and use in therapy are described.

In accordance with the present invention, there is provided a carrier chimeric protein having the following structure:

S1-A-S2-B,

wherein:

(a) S1 and S2 are spacers;

(b) A is a multimerization domain of a multimeric protein with a coiled coil domain (e.g., dimeric, trimeric, tetrameric or pentameric protein, or analogous products); and

(c) B is a protein- or amino acid-drug.

A protein- or amino acid-drug means any peptide-, polypeptide-, protein-drug or conjugate such as a thrombolytic agent (e.g., streptokinase, urokinase, single chain urokinase-like plasminogen activator, prourokinase, or derivatives). The spacers, S1 and S2, are sequences naturally occurring or not in the protein with coiled coil domain chosen to make the chimeric protein. The spacers S1 and S2 could be any amino acid sequence of between 0 and 300 amino acids, and will or not have the same amino acid sequence. These chimeric proteins or analogous products are useful in therapy.

Controllable drug-stability, drug-activity and drug-carrier are three aspects of the present invention. According to one aspect of the present invention, a new method for attaching a protein- or an amino acid-drug, to the multimerization domain of a multimeric protein with a coiled coil domain. More specifically, the invention utilizes the fact that the multimerization domain of a multimeric protein with a coiled coil domain (e.g., dimeric, trimeric, tetrameric or pentameric protein, or analogous products) can be used as a carrier for any peptide-, polypeptide-, or protein-drug. The multimerization domain of a multimeric protein with a coiled coil domain can be of variable length, depending on the requirements of the application.

In accordance with the present invention, there is also provided an isolated nucleic acid sequence encoding a chimeric protein, a vector comprising the nucleic acid sequence, and a host cell comprising such a vector.

In accordance with the present invention, there is also provided a targeted carrier chimeric protein, having the following structure:

T-S1-A-S2-B,

wherein:

(a) T is a specific amino acid domain to a specific site of action;

(b) S1 and S2 are spacers;

(c) A is the multimerization domain of any multimeric protein with a coiled coil domain (e.g., dimeric, trimeric, tetrameric or pentameric protein, or analogous products); and

(d) B is a protein- or an amino acid-drug.

Such chimeric proteins, comprising an active drug, a specific amino acid domain to a specific site of action, and a carrier, are useful as a means of delivering the drug to a specific site of action. The spacers, S1 and S2, are sequences naturally occurring or not in the protein with coiled coil domain chosen to make the chimeric protein. The spacers S1 and S2 could be any amino acid sequence of between 0 and 300 amino acids, and will or not have the same amino acid sequence. These chimeric proteins or analogous products are useful in therapy.

Controllable drug-specificity, drug-stability, drug-activity and drug-carrier are four aspects of the present invention. According to one embodiment of the present invention, there is provided a new method for attaching a protein- or amino acid drug to the multimerization domain of a multimeric protein with a coiled coil domain. A protein- or amino acid-drug, means any peptide-, polypeptide-, protein-drug or conjugate such as a thrombolytic agent (e.g., streptokinase, urokinase, staphylokinase, single chain urokinase-like plasminogen activator and prourokinase, or derivatives or fragments thereof). More specifically, the invention utilizes the fact that the multimerization domain of any multimeric protein with a coiled coil domain (e.g., dimeric, trimeric, tetrameric or pentameric protein, or analogous products) can be used as a carrier for any peptide-, polypeptide-, or protein-drug. It is an additional feature of the invention that, because an amino acid motif specific for a site of action of the active-drug-peptide, the chimeric protein product acts as a targeting agent. The product of the invention may usefully have other bound active agents. Such agents will be chosen with regard to the site of action, and to the nature of the problem that is addressed.

The invention further includes isolated nucleic acid sequences encoding a chimeric protein, a vector comprising these nucleic acid sequences, and host cells comprising such a vector.

In the chimeric proteins or analogous products described hereinabove, the multimerization domain of a multimeric protein with a coiled coil domain can be of variable length, depending on the requirements of the application. The multimerization domain of thrombospondin (TSP) protein (e.g., TSP-1 or TSP-5 (or COMP)) and other such glycoproteins (including matrilin, laminin, tenascin, collectin, and collagen, among others) can be used to make these chimeric proteins described herein. For example, a trimeric protein could be TSP-1 or TSP-2 and a pentameric protein could be TSP-3, TSP-4 or TSP-5 (or COMP).

According to another embodiment of the invention described herein, by inserting as much as desired of the active-drug-peptide, chimeric proteins had the utility of increasing the activity of the protein- or amino acid-drug. For examples: if the multimerization domain of the protein is trimeric (e.g., TSP-1 and TSP2), the number of inserted protein- or amino acid-drug could be a factor of 3 (3, 6, 9, 12, etc), and if the multimerization domain of the protein is pentameric (e.g., TSP-3, TSP-4 and TSP-5 (or COMP)), the number of inserted protein- or amino acid-drug could be a factor of 5 (5, 10, 15, etc).

The invention further includes isolated nucleic acid sequences encoding a chimeric protein as described herein, vectors comprising these nucleic acid sequences, and host cells comprising such a vector. In one embodiment, the invention comprises polynucleotides or nucleic acid molecules that encode the carrier chimeric protein described above. The present invention further encompasses gene therapy methods whereby DNA sequences, encoding carrier chimeric proteins, targeted carrier chimeric proteins described herein or conjugates are introduced into a patient. The selection of the appropriate gene therapy methods to treat a disease will be apparent to one skilled in the art.

For use as therapeutic agents, the chimeric protein of the present invention may be administered as is, or mixed with any pharmaceutically suitable carrier known to those of ordinary skill in the art. Binding of the chimeric protein to the carrier may be non-chemical, e.g., by adsorption or chemical, e.g., using a linker. The amount of the product administered will be determined largely to the severity of the condition to be treated. Such carrier chimeric protein product comprises a carrier bound thereto, without affecting (decreasing) its activity and specificity.

The multimerization domain of thrombospondin (TSP) protein (e.g., TSP-1 or TSP-5 (also named cartilage oligomeric matrix protein (COMP)) and other such glycoproteins with a coiled coil domain (including matrilin, laminin, tenascin, collectin, and collagen, among others) can be used to make the chimeric protein of the present invention. Such conjugates can be made by a conjugation of the multimerization domain of TSP-1 or TSP-5, and any protein- or amino acid-drug. Such protein- or amino acid-drug will be chosen with regard to the site of action, and to the nature of the problem that is addressed.

In one embodiment, the invention comprises polynucleotides or nucleic acid molecules that encode chimeric proteins having portions whose amino acid sequences are derived from human TSP-1.

In another embodiment, the invention comprises polynucleotides or nucleic acid molecules that encode chimeric proteins having portions whose amino acid sequence are derived from human TSP-5. The polynucleotides of the invention can be made by recombinant methods, can be made synthetically, can be replicated by enzymes in vitro (e.g., PCR) or in vivo system (e.g., by suitable host cells, when inserted into a vector appropriate for replication within the host cells), or can be made by a combination of methods. The polynucleotides of the invention can include DNA and its RNA counterpart. As used herein, “nucleic acid”, “nucleic acid molecule”, “nucleic acid sequence”, “oligonucleotide” and “polynucleotide” include DNA and RNA and chemical derivatives thereof, including phosphorothioate derivatives and RNA and DNA molecules having a label such as a radioactive isotope or a chemical adduct such as a fluorophore, chromophore or biotin. The RNA counterpart of a DNA is a polymer of ribonucleotides units, wherein the nucleotide sequence can be depicted as having the base U (uracil) at sites within a molecule where DNA has the base T (thymidine).

Isolated nucleic acid molecules or polynucleotides can be purified from a natural source or can be made recombinantly. Polynucleotides referred to herein as “isolated” are polynucleotides purified to a state beyond that in which they exist in cells. They include polynucleotides obtained by methods described herein, similar methods or other suitable methods, and also include essentially pure polynucleotides produced by chemical synthesis or by combination of biological and chemical methods, and recombinant polynucleotides that have been isolated. The term “isolated” are used herein for nucleic acid molecules, indicates that the molecule in question exists in a physical milieu distinct from that in which it occurs in nature. For example, an isolated polynucleotide may be substantially isolated with respect to the complex cellular milieu in which it naturally occurs, and may even be purified essentially to homogeneity, for example as determined by agarose or polyacrylamide gel electrophoresis or by A260/A280 measurements, but may also have further cofactors or molecular stabilizers (for instance, buffers or salts) added.

In accordance with the present invention, there is also provided a method for producing the chimeric protein of the present invention or a variant thereof, and expression systems and host cells containing a vector appropriate for expression of the chimeric protein of the present invention. Variants of the chimeric protein include those having amino acid sequences that differ from those sequences described herein wherein those variants have several, such as 5 to 10, 1 to 5, or 3, 2 or 1 amino acids substituted, deleted, or added, in any combination, compared to the sequences described herein.

In one embodiment, variants may have silent substitutions, additions and deletions that do not alter the properties and activities of the chimeric protein. Variants can also be modified polypeptides in which one or more amino acid residues are modified, and mutants comprising one or more modified residues. Proteins and polypeptides described herein can be assessed for their activities by using an assay such as those known in the art.

Cells that express such a chimeric protein or a variant thereof can be made and maintained in culture, under conditions suitable for expression, to produce protein for isolation. These cells can be prokaryotic or eukaryotic. Examples of prokaryotic cells that can be used for expression (as “host cells”; “cell” including herein cells of tissues, cell cultures, cell strains and cell lines) include Escherichia coli, Bacillus subtilis and other bacteria. Examples of eukaryotic cells that can be used for expression include yeast cells such as Saccharomyces cerevisiae, Schizosaccharomyces pombe, Pichia pastoris and other lower eukaryotic cells, and cells of higher eukaryotes such as those from insects and mammals. Suitable cells of mammalian origin include primary cells, and cell lines such as CHO, HeLa, 3T3, BHK, COS, 293, and Jurkat cells. Suitable cells of insect origin include primary cells, and cell lines such as SF9 and High five cells. (See, e.g., Ausubel F M et al., eds. Current Protocols in Molecular Biology, Greene Publishing Associates and John Wiley & Sons Inc., (containing up through 1998)).

In one embodiment, host cells that produce a recombinant chimeric protein, variant, or portions thereof can be made as follows. A gene encoding a chimeric protein described herein can be inserted into a nucleic acid vector, e.g., DNA vector, such as a plasmid, virus or other suitable replicon (including vectors suitable for use in gene therapy, such as those derived from adenovirus or others; see, for example Xu et al., (Molecular Genetics and Metabolism, 1998, 63: 103-109)). The gene encoding the chimeric protein can be present in a single copy or multiple copies, or the gene can be integrated in a host cell chromosome. A suitable replicon or integrated gene can contain all or part of the coding sequence for the protein or variant, operably linked to one or more expression control regions whereby the coding sequence is under the control of transcription signals and linked to appropriate translation signals to permit translation. The vector can be introduced into cells by a method appropriate to the type of host cells (e.g., transformation, electroporation, and infection). For expression from the gene, the host cells can be maintained under appropriate conditions (e.g., in the presence of inducer, normal growth conditions, etc.). Proteins or polypeptides thus produced can be recovered (e.g., from the cells, the periplasmic space, culture medium) using suitable techniques.

The invention also relates to isolated proteins or polypeptides encoded by nucleic acids of the present invention. Isolated proteins can be purified from a natural source or can be made recombinantly. Proteins or polypeptides referred to herein as “isolated” are proteins or polypeptides purified to a state beyond that in which they exist in cells and include proteins or polypeptides obtained by methods described herein, similar methods or other suitable methods, and also include essentially pure proteins or polypeptides, proteins or polypeptides produced by chemical synthesis or by combinations or biological and chemical methods, and recombinant proteins or polypeptides which are isolated. Thus, the term “isolated” as used herein, indicates that the polypeptide in question exists in a physical milieu distinct from the cell in which its biosynthesis occurs. For example, an isolated (1) carrier chimeric proteins, having the following structure: S1-A-S2-B, wherein: (a) S1 and S2 are spacers; (b) A is the multimerization domain of TSP-1 or TSP-5; and (c) B is a protein- or an amino acid-drug, and (2) targeted carrier chimeric proteins, having the following structure: T-S1-A-S2-B, wherein: (a) T is a specific amino acid domain to a specific site of action (b) S 1 and S2 are spacers; (c) A is the multimerization domain of TSP-1 or TSP-5; and (d) B is a protein- or an amino acid-drug, may be purified essentially to homogeneity, for example as determined by PAGE or column chromatography (for example, HPLC), but may also have further cofactors or molecular stabilizers added to the purified protein to enhance activity. In one embodiment, proteins or polypeptides are isolated to a state at least about 75% pure; more preferably at least about 85% pure, and still more preferably at least about 95% pure, as determined by Coomassie blue staining of proteins on SDS-polyacrylamide gels.

Chimeric or fusion proteins can be produced by a variety of methods. For example, a chimeric protein can be produced by the insertion of a chimeric protein gene or portion thereof into a suitable expression vector, such as Bluescript SK+/(Stratagene), pGEX-4T-2 (Pharmacia), pET-15b, pET-20b(+) or pET-24(+) (Novagen). The resulting construct can be introduced into a suitable host cell for expression. Upon expression, chimeric protein can be purified from a cell lysates by means of suitable affinity matrix (see, e.g., Current Protocols in Molecular Biology (Ausubel F M et al., eds., Vol. 2, pp. 16.4.1-16.7.8, containing supplements up through Supplement 44, 1998).

Polypeptides of the invention can be recovered and purified from cell cultures by well-known methods. The recombinant protein can be purified by ammonium sulfate precipitation, heparin-Sepharose affinity chromatography, gel filtration chromatography and/or sucrose gradient ultracentrifugation using standard techniques. Further methods that can be used for purification of the polypeptide include ethanol precipitation, acid extraction, anion or cation exchange chromatography, phosphocellulose chromatography, hydrophobic interaction chromatography, affinity chromatography, hydroxylapatite chromatography and high performance liquid chromatography. Known methods for refolding protein can be used to generate active conformation if the polypeptide is denatured during isolation or purification.

The present invention further encompasses gene therapy methods whereby DNA sequences encoding carrier chimeric proteins, targeted carrier chimeric proteins or conjugates are introduced into a patient. The selection of the appropriate gene therapy methods to treat a disease will be apparent to one skilled in the art.

The thrombospondins (TSPs) are a family of extracellular proteins that participate in cell-to-cell and cell-to-matrix communications (Lawler, Curr Opin in Cell Biol, 2000, 12: 634-640). Five family members (TSP-1, TSP-2, TSP-3, TSP-4 and TSP-5 (or COMP)), each representing a separate gene product, exist in most vertebrate species. Many tissues, such as the heart, cartilage and brain, express most of the TSP gene products. The TSPs appear to function at the cell surface to bring together membrane proteins and cytokines that regulate extracellular structure and cellular phenotype (Lawler, Curr Opin in Cell Biol, 2000, 12: 634-640). Like most large extracellular proteins, the TSPs are composed of several structural domains. TSP gene family can be divided into two subgroups on the basis of their architecture. TSP-1 and TSP-2 are similar in terms of their molecular architecture and have been designated subgroup A TSPs. TSP-3, -4 and -5 are also similar to each other and are distinct from TSP-1 and TSP-2, these TSPs comprise group B. Whereas the subgroup A TSPs are trimers, the subgroup B TSPs are pentamers.

TSP-1 was the first family member to be identified and it is a major constituent of human blood platelets. TSP-1 is a 450 kDa homotrimeric matricellular glycoprotein that regulates attachment, proliferation, migration, and differentiation of various cell types (Bornstein, J Cell Biol, 1995, 130: 503-506). It appears that the function of TSP-1 is to direct the formation of multiprotein complexes that modulate cellular phenotype in much the same way that the formation of multiprotein complexes regulate cell adhesion, signal transduction and transcriptions (Lawler, Curr Opin in Cell Biol, 2000, 12: 634-640). TSP-5 (or COMP) is the most recent addition to the family of TSPs. TSP-5 is a 520 kDa pentameric glycoprotein in which multimerization appears to be directed by α-helical segments that come (in the amino acid sequence) either before or after the cysteine residues that form the interaction disulfide bonds.

The multimerization domain of TSP protein (e.g., TSP-1 or TSP-5) and other such glycoproteins with a coiled coil domain (including matrilin, laminin, tenascin, collectin, and collagen, among others) can be used to make the chimeric protein of the present invention. Such conjugates can be made by a conjugation of the multimerization domain of TSP-1 or TSP-5, and any protein- or amino acid-drug. Such protein- or amino acid drug will be chosen with regard to the site of action, and to the nature of the problem that is addressed. The invention comprises polynucleotides or nucleic acid molecules that encode chimeric proteins having portions whose amino acid sequences are derived from human TSP-1 or TSP-5. The TSP-1 assembly domain spontaneously forms a 3-stranded α-helical domain, allowing for the use of the TSP-1 domain as a trimerization tool. The TSP-5 assembly domain spontaneously forms a 5-stranded α-helical domain, allowing for the use of the TSP-5 domain as a pentamerization tool. Thus carrier chimeric proteins and targeted carrier chimeric proteins using the multimerization domain of TSP-1 or TSP-5 in accordance with the present invention are expected to be correctly folded and multimeric so that they better mimic the drug with an increase of its stability and activity. If the protein- or amino acid-drug is derived from human proteins, these chimeric proteins, derived from portions of human proteins, should not be immunogenic in humans. The invention further includes isolated nucleic acids encoding any of the above chimeric proteins, vectors comprising these nucleic acids, and host cells comprising any of said vectors.

The method to construct genes encoding either the carrier chimeric proteins or the targeted carrier chimeric proteins as defined previously can be applied more broadly to produce polynucleotides, and vectors and host cells comprising such polynucleotides, wherein the polynucleotides encode the multimerization domain of a protein with coiled coil domain. A protein with coiled coil domain may include matrilin, laminin, tenascin, and collagen, among others. In each case, a portion of a polynucleotide known to encode full-length of a drug (e.g., a thrombolytic agent, human endostatin, angiostatin, platelet factor 4 (GeneBank Accession No. M25897) or prolactin (GeneBank Accession No. V00566)), can be chosen for cloning into the multimerization domain cDNA of a protein with coiled coil domain as illustrated herein for TSP-1 or TSP-5.

The present invention further encompasses gene therapy methods whereby DNA sequences encoding carrier chimeric proteins, targeted carrier chimeric proteins or conjugates are introduced into a patient. The selection of the appropriate gene therapy methods to treat a disease will be apparent to one skilled in the art.

The present invention will be more readily understood by referring to the following examples, which are given to illustrate the invention rather than to limit its scope.

### EXAMPLE I

### Carrier Chimeric Proteins Using the Multimerization Domain of TSP-1

If the multimerization domain of the carrier chimeric proteins is from TSP-1, the carrier chimeric proteins will have the following structure:

S1-A-S2-B,

wherein: S 1 and S2 are spacers; A is the multimerization domain of TSP-1; and B is a protein- or amino acid-drug.

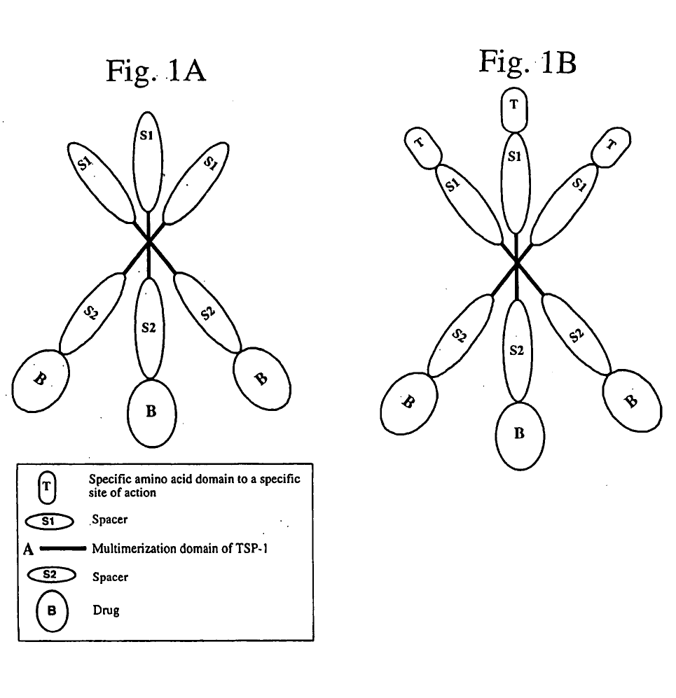

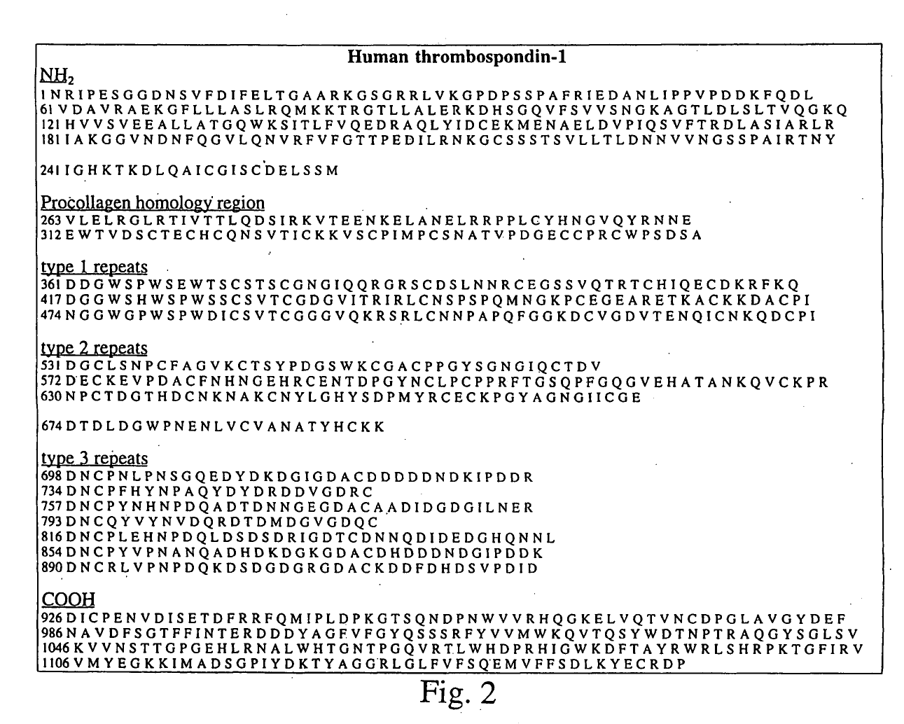

The assembled protein is a trimer containing 3 copies of the drug (see FIG. 1A). The spacers, S1 and S2 are sequences naturally occurring or not in TSP-1. The spacers S1 and S2 could be any amino acid sequence of between 0 and 300 amino acids, and S1 and S2 can have the same amino acid sequence or not. The spacers can be an amino acid, peptide or polypeptide, and can have enzymatic or binding activity of their own. In one case, the S1 spacer is absent and S2 spacer could be one of the type 1 repeats of TSP-1 (the first, the second or the third). In another case, the S1 spacer is absent and S2 spacer could one combination of two type 1 repeats of TSP-1 (the first and the second, the second and the third, or the first and the third). In another case, the S1 spacer is absent and S2 spacer could be all the three type 1 repeats of TSP-1 (the first, the second and the third). By the genomic structure, the multimerization domain of TSP-1 are amino acid residues 241-360, which include the procollagen homology region of TSP-1 (amino acid residues 263-360), the type 1 repeats of TSP-1 are amino acid residues 361-416 (first), amino acid residues 417-473 (second), and 474-530 (third) (see FIG. 2). If amino acid sequences that activate TGF-βare included in the product protein, and are found to reduce drug-activity, the RFK sequence can be mutated (to QM for example) to a sequence that does not activate TGF-β, by appropriate manipulations of the nucleic acid molecule or construct encoding the chimeric proteins. In another case, the chimeric proteins encoded by the polynucleotides of the invention are variants of the immediately aforementioned chimeric protein which have activity that is similar in quality and quantity (for example, plus or minus one order of magnitude in an assay) to the drug activity of the protein whose amino acid sequence are described above. The invention further includes isolated nucleic acid molecules encoding any of the above chimeric proteins, vectors comprising these nucleic acid molecules, and host cells comprising any of said vectors. The present invention further encompasses gene therapy methods whereby DNA sequences encoding the carrier chimeric protein or a conjugate thereof are introduced into a patient. The selection of the appropriate gene therapy methods to treat a disease will be apparent to one skilled in the art.

### EXAMPLE II

### Targeted Carrier Chimeric Proteins Using the Multimerization Domain of TSP-1

If the multimerization domain of the targeted carrier chimeric proteins is from TSP-1, the targeted carrier chimeric proteins will have the following structure:

T-S1-A-S2-B,

wherein: T is a specific amino acid domain to a specific site of action; S1 and S2 are spacers; A is the multimerization domain of TSP-1; and B is a protein- or amino acid-drug (see FIG. 1B). The assembled protein is a trimer containing 3 copies of the drug, and 3 copies of the specific amino acid domain to a specific site of action. The spacers, S1 and S2, are sequences naturally occurring or not in TSP-1. The spacers S1 and S2 could be any amino acid sequence of between 0 and 300 amino acids, and will or not have the same amino acid sequence. The spacers can be an amino acid, peptide or polypeptide, and can have enzymatic or binding activity of their own. In one embodiment, the spacer S1 could be any amino acid sequence of between 0 and 300 amino acids, and the S2 spacer preferably could be one of the type 1 repeats of TSP-1 (the first, the second or the third). In another embodiment, the S1 spacer could be any amino acid sequence of between 0 and 300 amino acids and the S2 spacer could be one combination of two type 1 repeats of TSP-1 (the first and the second, the second and the third, or the first and the third). In another embodiment, the S1 spacer could be any amino acid sequence of between 0 and 300 amino acids and the S2 spacer could be all the three type 1 repeats of TSP-1 (the first, the second and the third). By the genomic structure, the multimerization domain of TSP-1 are amino acid residues 241-360, which include the procollagen homology region of TSP-1 (amino acid residues 263-360), the type 1 repeats of TSP-1 are amino acid residues 361-416 (first), amino acid residues 417-473 (second), and 474-530 (third) (see FIG. 2). If amino acid sequences that activate TGF-β are included in the product protein, and are found to reduce drug-activity, the RFK sequence can be mutated (to QM for example) to a sequence that does not activate TGF-β, by appropriate manipulations of the nucleic acid molecule or construct encoding the chimeric proteins. In another case, the chimeric proteins encoded by the polynucleotides of the invention are variants of the immediately aforementioned chimeric protein which have activity that is similar in quality and quantity (for example, plus or minus one order of magnitude in an assay) to the drug activity of the protein whose amino acid sequence are described above. The invention further includes isolated nucleic acid molecules encoding a chimeric protein of the present invention, vectors comprising the nucleic acid molecules, and host cells comprising such vectors. The present invention further encompasses gene therapy methods whereby DNA sequences encoding targeted carrier chimeric proteins or conjugates are introduced into a patient. The selection of the appropriate gene therapy methods to treat a disease will be apparent to one skilled in the art.

### EXAMPLE III

### Carrier Chimeric Proteins Using the Multimerization Domain of TSP-5

If the multimerization domain of the carrier chimeric proteins is from TSP-5, the carrier chimeric proteins of the present invention will have the following structure:

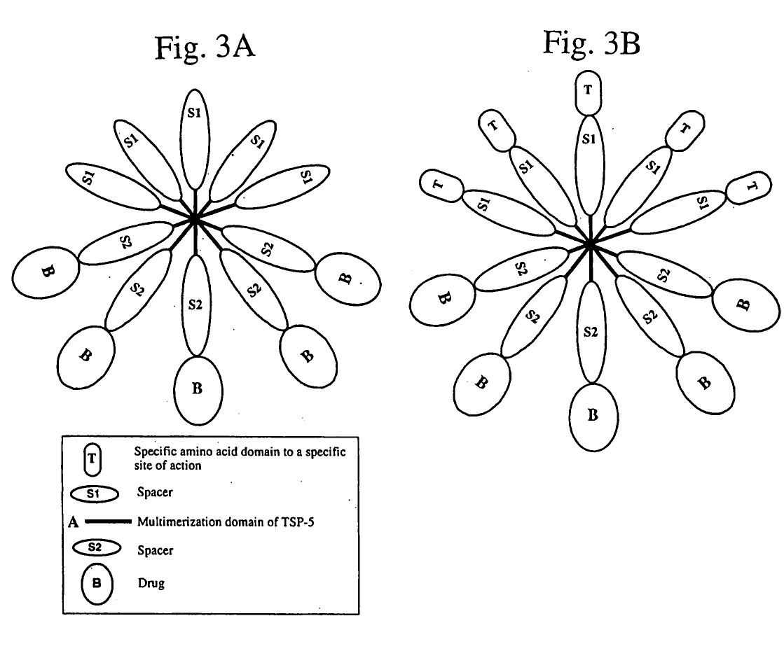

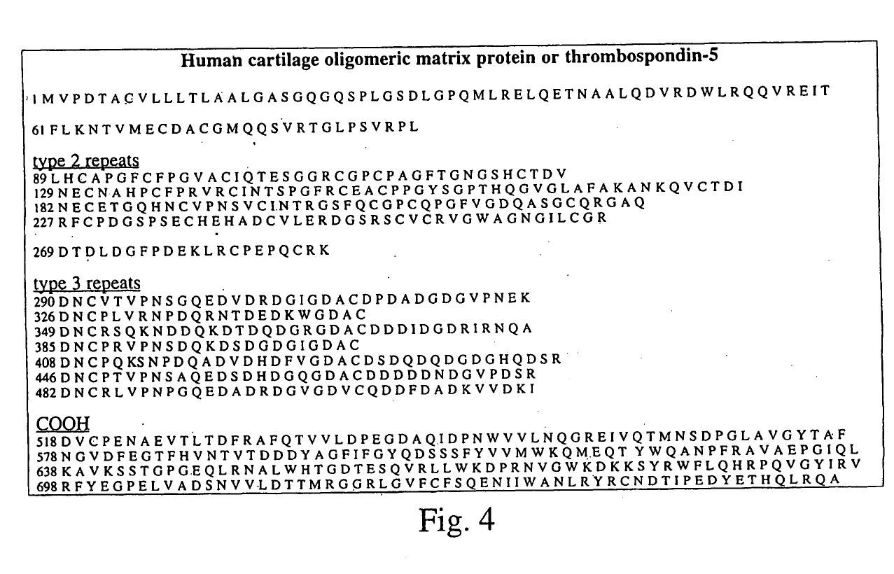

S1-A-S2-B,

wherein: S1 and S2 are spacers; A is the multimerization domain of TSP-5; and B is a protein- or amino acid-drug (see FIG. 3A). The assembled protein is a pentamer containing 5 copies of the drug. The spacers, S1 and S2, are sequences naturally occurring or not in TSP-5. The spacers S1 and S2 could be any amino acid sequence of between 0 and 300 amino acids, and will or not have the same amino acid sequence. The spacers can be an amino acid, peptide or polypeptide, and can have enzymatic or binding activity of their own. In one case, the S1 spacer is absent and S2 spacer preferably could be the first type 2 repeat of human TSP-5. By the genomic structure, the multimerization domain of TSP-5 are amino acid residues 1-88, and the first type 2 repeat of TSP-5 are amino acid residues 89-128, whereas the other type 2 repeat of TSP-5 are amino acid residues 129-181 (second), 182-226 (third) and 227-268 (fourth) (see FIG. 4). In another embodiment, the chimeric proteins encoded by the polynucleotides of the invention are variants of the immediately aforementioned chimeric protein which have activity that is similar in quality and quantity (for example, plus or minus one order of magnitude in an assay) to the drug activity of the protein whose amino acid sequence are described above. The invention further includes isolated nucleic acids encoding any of the above chimeric proteins, vectors comprising these nucleic acids, and host cells comprising any of said vectors. The present invention further encompasses gene therapy methods whereby DNA sequences encoding carrier chimeric proteins or conjugates are introduced into a patient. The selection of the appropriate gene therapy methods to treat a disease will be apparent to one skilled in the art.

### EXAMPLE IV

### Targeted Carrier Chimeric Proteins Using the Multimerization Domain of TSP-5

If the multimerization domain of the targeted carrier chimeric proteins is from TSP-5, the targeted carrier chimeric proteins will have the following structure:

T-S1-A-S2-B,

wherein: T is a specific amino acid domain to a specific site of action; S1 and S2 are spacers; A is the multimerization domain of TSP-5; and B is a protein- or amino acid-drug (see FIG. 3B). The assembled protein is a pentamer containing 5 copies of the drug, and 5 copies of the specific amino acid domain to a specific site of action. The spacers, S1 and S2, are sequences naturally occurring or not in TSP-5. The spacers S1 and S2 could be any amino acid sequence of between 0 and 300 amino acids, and will or not have the same amino acid sequence. The spacers can be an amino acid, peptide or polypeptide, and can have enzymatic or binding activity of their own. In one embodiment, the spacer S1 could be any amino acid sequence of between 0 and 300 amino acids, and the S2 spacer preferably could be the first type 2 repeat of human TSP-5. By the genomic structure, the multimerization domain of TSP-5 are amino acid residues 188, and the first type 2 repeat of TSP-5 are amino acids residues 89-128 (see FIG. 4). In another embodiment, the chimeric proteins encoded by the polynucleotides of the invention are variants of the immediately aforementioned chimeric protein which have activity that is similar in quality and quantity (for example, plus or minus one order of magnitude in an assay) to the drug activity of the protein whose amino acid sequence are described herein. The invention further includes isolated nucleic acids encoding any of the above chimeric proteins, vectors comprising these nucleic acids, and host cells comprising any of said vectors. The present invention further encompasses gene therapy methods whereby DNA sequences targeted carrier chimeric proteins or conjugates are introduced into a patient. The selection of the appropriate gene therapy methods to treat a disease will be apparent to one skilled in the art.

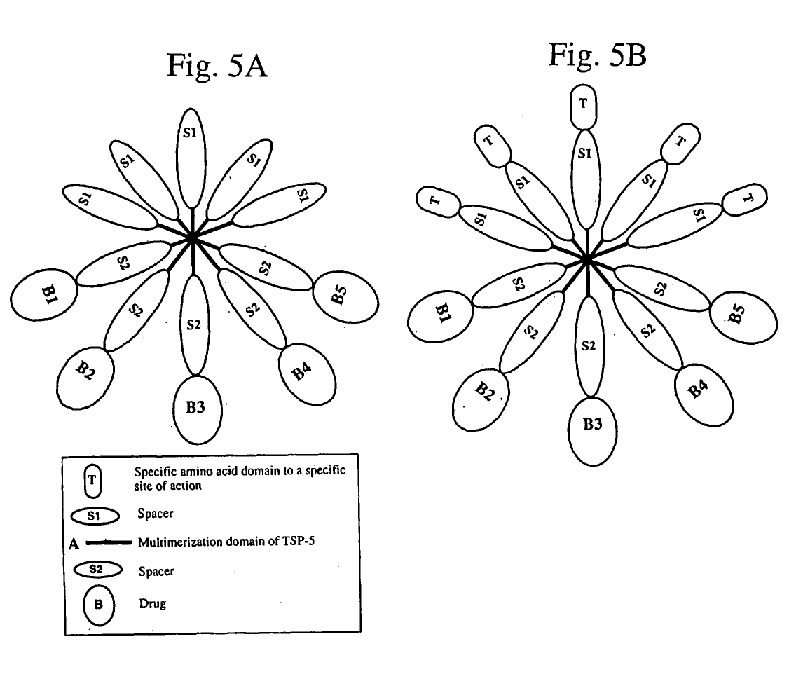

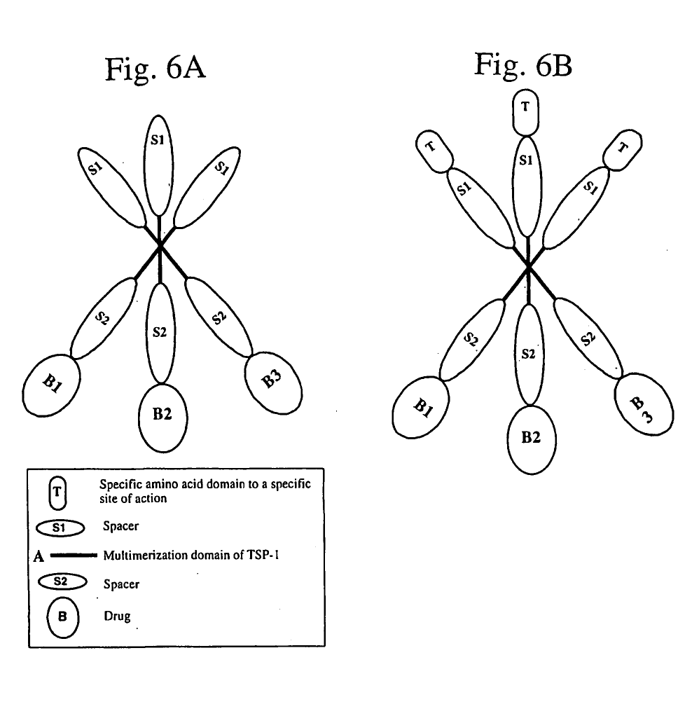

In addition, a portion of different drugs, named drug 1, drug 2, drug 3, drug 4, and drug 5 for example wherein that portion of each drug encodes a polypeptide having the same or different activities can be added to or incorporated into a DNA construct encoding: (1) carrier chimeric proteins, having the following structure: S1-A-S2-drug 1, wherein: (a) S1 and S2 are spacers; (b) A is the multimerization domain of TSP-5, and (2) targeted carrier chimeric proteins, having the following structure: T-S1-A-S2-drug 1, wherein: (a) T is a specific amino acid domain to a specific site of action; (b) S1 and S2 are spacers; and (c) A is the multimerization domain of TSP-5, such that drug 1 derived polypeptide and a polypeptide derived from drug 2, drug 3, drug 4, or drug 5 are produced fused together in tandem on the same “arm” of the “5-armed” TSP-5-multimerized pentamer (see FIG. 5). Different expression constructs can be introduced into the same host cells such that two or more chimeric protein “arms” of different types are joined at the TSP-5 multimerization domain. The same principle could be applied if the multimerization domain is from TSP-1, thus different expression constructs can be introduced into the same host cells such that two or more chimeric protein “arms” of different types are joined at the TSP-1 multimerization domain (see FIG. 6).

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A carrier chimeric protein of the following formula:
S1-A-S2-B,
wherein:
(a) S1 is present or absent and is a spacer;
(b) S2 is a spacer;
(c) A is a multimerization domain of a multimeric protein with a coiled coil domain (e.g., dimeric, trimeric, tetrameric or pentameric protein, or analogous products); and
(d) B is a protein- or amino acid-drug,
when S1 is present, S1 and S2 are identical or different.

2. A carrier chimeric protein of the following formula:
T-S1-A-S2-B,
wherein:
(a) T is a specific amino acid domain to a specific site of action;
(b) S1 and S2, identical or different, are spacers;
(c) A is the multimerization domain of any multimeric protein with a coiled coil domain (e.g., dimeric, trimeric, tetrameric or pentameric protein, or analogous products); and
(d) B is a protein- or amino acid-drug.

3. A carrier chimeric protein as defined in claim 1 or 2, wherein S1 is an amino acid sequence of 0 to 300 amino acids in length.

4. A carrier chimeric protein as defined in claim 1 or 2, wherein S2 is an amino acid sequence of 0 to 300 amino acids in length.

5. A carrier chimeric protein as defined in claim 1 or 2, wherein S2 comprises a type 1 repeat of human TSP-1, with or without the TGF-βactivation region of human TSP-1.

6. A carrier chimeric protein as defined in claim 1 or 2, wherein S2 comprises two type 1 repeats of human TSP-1, with or without the TGF-βactivation region of human TSP-1.

7. A carrier chimeric protein as defined in claim 1 or 2, wherein S2 comprises three type 1 repeats of human TSP-1, with or without the TGF-βactivation region of human TSP-1.

8. A carrier chimeric protein as defined in claim 1 or 2, wherein S2 comprises the first type 2 repeats of TSP-5.

9. A carrier chimeric protein as defined in claim 1 or 2, wherein A is the multimerization domain of TSP-1, TSP-2, TSP-3, TSP-4 or TSP-5.

10. A carrier chimeric protein as defined in claim 1 or 2, wherein B is a protein-drug or an amino acid-drug selected from the group consisting of a peptide-drug, a polypeptide-drug, a protein-drug or a conjugate thereof.

11. A carrier chimeric protein as defined in claim 10, wherein B is a thrombolytic agent, and more particularly a streptokinase, an urokinase, a single chain urokinase-like plasminogen activator, a prourokinase, or a derivative thereof.

12. A nucleic acid molecule encoding a carrier chimeric protein as defined in any one of claims 1 to 11.

13. A vector comprising the nucleic acid molecule of claim 12.

14. A host cell comprising the vector of claim 13.

15. A method for producing a carrier chimeric protein, said method comprising maintaining the host cell of claim 14 under conditions suitable for expression of said nucleic acid molecule, whereby said carrier chimeric protein is produced.
